(19) United States
(12) Patent Application Publication
(10) Pub. No.: US 2026/0283785 A1
(43) Pub. Date: Sep. 24, 2026

(54) Intraocular Lens Design Generator for Mechanical Stability

(71) Applicant: Alcon Inc., Fribourg (CH)

(72) Inventors: Sonalee Tambat, Irvine, CA (US); Manish Malhar Agarkar, Irvine, CA (US)

(21) Appl. No.: 19/567,486

(22) Filed: Mar. 16, 2026

Related U.S. Application Data
(60) Provisional application No. 63/774,828, filed on Mar. 20, 2025.

(51) Int. Cl. A61F 2/16; G06F 30/27

(52) U.S. Cl. CPC A61F 2/1613; G06F 30/27; A61F 2240/002

(57) ABSTRACT

An intraocular lens design generator for mechanical stability is described. An IOL design iteration may be generated based at least on input mechanical stability criteria using a shape morphing engine. A performance of the IOL design iteration with respect to the input mechanical stability criteria may be predicted using a machine learning model trained on historical IOL design data and corresponding historical simulation data. An IOL design may be output based on the predicted performance of the IOL design iteration.

### BACKGROUND

Intraocular lenses (IOLs) are artificial lens implants used to replace an eye's natural lens, typically during cataract surgery or to correct refractive errors. Typically, IOL design and development is aimed at improving visual outcomes and patient comfort following lens implantation. Current IOL designs vary in their materials, optical properties, and mechanical characteristics. The mechanical stability of an IOL may affect its performance, as it affects the lens's position within the eye and its ability to maintain focus. Traditional approaches to IOL design often involve iterative processes of geometric modifications, prototyping, and clinical testing, which can be time-consuming and resource intensive.

### SUMMARY

An intraocular lens design generator for mechanical stability is described. An IOL design iteration may be generated based at least on input mechanical stability criteria using a shape morphing engine. A performance of the IOL design iteration with respect to the input mechanical stability criteria may be predicted using a machine learning model trained on historical IOL design data and corresponding historical simulation data. An IOL design may be output based on the predicted performance of the IOL design iteration.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures. Entities represented in the figures are indicative of one or more entities and thus, reference is made interchangeably to single or plural forms of the entities in the discussion.

FIG. 1 is an illustration of an environment in an example implementation that is operable to employ an intraocular lens design generator for mechanical stability as described herein.

FIG. 2 depicts a cross-section of an eye.

FIG. 3 depicts a top view of an intraocular lens with a bench test setup.

FIG. 4 depicts a side view of the intraocular lens with a bench test setup shown in FIG. 3.

FIG. 5 depicts an example training process that may be used in generating the deep learning model of the IOL design module of FIG. 1.

FIG. 6 depicts an example implementation of using the deep learning model to design an intraocular lens based on desired mechanical stability criteria.

FIG. 7 depicts an example procedure in which a deep learning model is trained to predict intraocular lens device performance based on geometry.

FIG. 8 depicts an example procedure in which new intraocular lens designs are generated using a deep learning model.

FIG. 9 illustrates an example system including various components of an example device that can be implemented as any type of computing device as described and/or utilized with reference to FIGS. 1-8 to implement the techniques described herein.

### DETAILED DESCRIPTION

### Overview

The development of intraocular lenses (IOLs) that maintain consistent mechanical stability across a range of patient anatomies and surgical conditions presents ongoing challenges. Factors such as capsular bag size, zonular support, and post-operative healing can all influence a stability of the IOL. Additionally, the complex interplay between an IOL's geometric parameters and its mechanical behavior makes predicting performance based solely on design specifications difficult.

Traditional approaches to IOL design often involve iterative processes of geometric modifications, physical prototyping, and testing. This conventional method is time-consuming, resource-intensive, and limits the exploration of diverse design possibilities. As the demand for more advanced and personalized IOL solutions grows, there is an increasing need for efficient and accurate methods to optimize IOL designs for mechanical stability. Physics-based computational modeling and simulation techniques have been employed to aid in this process, but these approaches often include solving complex equations to evaluate new designs. These techniques use significant time and computational resources.

Accordingly, an intraocular lens design generator for mechanical stability is described herein. By way of example, intraocular lens design generator leverages machine learning techniques to rapidly explore and optimize IOL designs, which may significantly reduce the time and resources involved compared to traditional methods and conventional computational simulations.

In accordance with the described techniques, the IOL design generator combines a trained deep learning model with a shape morphing engine. The deep learning model, which is trained on historical IOL design data and corresponding simulation results, may quickly predict the mechanical stability performance of new IOL designs without performing time-consuming physics-based simulations. The shape morphing engine enables efficient generation and modification of IOL designs based on these predictions.

This physics-based machine learning technique described herein enables the rapid generation and evaluation of multiple IOL design iterations with respect to mechanical stability performance, enabling iterative refinement of IOL designs without human intervention. The techniques described herein also may enable additional criteria to be considered, such as optical performance or manufacturing considerations, while significantly reducing design cycle time and computing resource usage compared to traditional methods. Moreover, the described techniques may enable more personalized IOL designs tailored to specific patient needs to be generated, which may improve outcomes for patients undergoing IOL implantation.

In some aspects, the techniques described herein relate to a system for generating an intraocular lens (IOL) design, including: an IOL design module implemented in a non-transitory computer-readable storage medium and configured to perform operations including: generate an IOL design iteration based at least on input mechanical stability criteria using a shape morphing engine; predict a performance of the IOL design iteration with respect to the input mechanical stability criteria using a machine learning model trained on historical IOL design data and corresponding historical simulation data; and output an IOL design based on the predicted performance of the IOL design iteration.

In some aspects, the techniques described herein relate to a system, wherein the IOL design iteration is further based on IOL geometric parameters including at least one of an optic diameter, an overall diameter, an edge thickness, a haptic angle, a surface curvature, or a baseline IOL design.

In some aspects, the techniques described herein relate to a system, wherein predicting the performance of the IOL design iteration includes: extracting three-dimensional geometric information of the IOL design iteration using a deep neural network of the machine learning model; and estimating a mechanical stability of the IOL design iteration based on the extracted three-dimensional geometric information using the machine learning model.

In some aspects, the techniques described herein relate to a system, wherein outputting the IOL design based on the predicted performance of the IOL design iteration includes: determining whether a performance threshold has been met based on the predicted performance; and in response to the performance threshold being met, outputting the IOL design iteration as the IOL design; or in response to the performance threshold not being met, adjusting the IOL design iteration via the shape morphing engine.

In some aspects, the techniques described herein relate to a system, wherein adjusting the IOL design iteration includes: modifying, via the shape morphing engine, at least one geometric parameter of the IOL design iteration; estimating, via the machine learning model, an updated predicted performance of the adjusted IOL design iteration; and determining whether the performance threshold has been met based on the updated predicted performance of the adjusted IOL design iteration.

In some aspects, the techniques described herein relate to a system, wherein the IOL design module is further configured to perform operations including: generating multiple IOL design iterations; predicting performances for each of the multiple IOL design iterations; and selecting an IOL design based on a comparison of the predicted performances.

In some aspects, the techniques described herein relate to a system, wherein the shape morphing engine employs topology modification algorithms to generate the IOL design iteration.

In some aspects, the techniques described herein relate to a system, wherein the corresponding historical simulation data include results from physics-based computational simulations for individual IOL designs of the historical IOL design data, and wherein the machine learning model predicts the performance of the IOL design iteration based on learned relationships between geometric features extracted from the historical IOL design data and the results of the physics-based computational simulations.

In some aspects, the techniques described herein relate to a system, wherein the IOL design module is further configured to perform operations including: predicting an optical performance of the IOL design using the machine learning model.

In some aspects, the techniques described herein relate to a method for generating intraocular lens (IOL) designs, including: training a deep learning model using historical IOL design data as an input and corresponding historical simulation data as an expected output; after training the deep learning model, receiving input parameters including at least mechanical stability criteria for a new IOL design; generating an IOL design iteration based on the input parameters using a shape morphing engine linked to the trained deep learning model; predicting a performance of the IOL design iteration with respect to the mechanical stability criteria using the trained deep learning model; iteratively refining the IOL design iteration using the shape morphing engine and based on the predicted performance; and outputting the new IOL design based on the iterative refinement.

In some aspects, the techniques described herein relate to a method, wherein the corresponding historical simulation data include results from physics-based computational simulations, and wherein the trained deep learning model predicts the performance of the IOL design iteration based on learned relationships between geometric features extracted from the historical IOL design data and the results of the physics-based computational simulations.

In some aspects, the techniques described herein relate to a method, wherein generating the IOL design iteration includes modifying an initial IOL design by adjusting, via the shape morphing engine automatically and without user intervention, at least one of an optic diameter, a haptic angle, an overall diameter, an edge thickness, or a surface curvature.

In some aspects, the techniques described herein relate to a method, wherein the deep learning model includes a neural network configured to extract geometric information from three-dimensional IOL designs.

In some aspects, the techniques described herein relate to a method, wherein iteratively refining the IOL design iteration using the shape morphing engine and based on the predicted performance includes: determining whether a performance threshold has been met based on the predicted performance; and in response to the performance threshold being met, selecting the IOL design iteration as the new IOL design; or in response to the performance threshold not being met: adjusting the IOL design iteration using the shape morphing engine; and repeating the predicting and adjusting until the performance threshold is met.

In some aspects, the techniques described herein relate to a method, wherein outputting the new IOL design includes generating a three-dimensional model of the new IOL design.

In some aspects, the techniques described herein relate to a method, wherein outputting the new IOL design includes providing parametric descriptions of a geometry of the new IOL design.

In some aspects, the techniques described herein relate to a method for generating an intraocular lens (IOL) design with enhanced mechanical stability, including: receiving historical IOL design data and corresponding historical simulation data; training a machine learning model using the historical IOL design data as an input and the corresponding historical simulation data as an expected output; receiving input parameters for a new IOL design, the input parameters including mechanical stability criteria; generating multiple IOL design iterations based on the input parameters using a shape morphing engine; predicting performances for each of the multiple IOL design iterations with respect to the mechanical stability criteria using the trained machine learning model; selecting an IOL design iteration from the multiple IOL design iterations based on the predicted performances; and outputting the selected IOL design iteration as the new IOL design.

In some aspects, the techniques described herein relate to a method, wherein the mechanical stability criteria include a maximum allowable displacement under compression, and wherein generating the multiple IOL design iterations includes modifying at least one of an optic diameter, a haptic angle, an overall diameter, an edge thickness, or a surface curvature of a previous IOL design iteration.

In some aspects, the techniques described herein relate to a method, wherein outputting the selected IOL design iteration as the new IOL design includes: generating a three-dimensional visualization of the new IOL design for display on a display device; and storing the new IOL design to a non-transitory memory.

In some aspects, the techniques described herein relate to a method, wherein selecting the IOL design iteration from the multiple IOL design iterations based on the predicted performances includes: ranking the multiple IOL design iterations with respect to at least the mechanical stability criteria; and selecting a best-ranked IOL design iteration as the new IOL design.

In the following discussion, an example environment is first described that may employ the techniques described herein. Example implementation details and procedures are then described which may be performed in the example environment as well as other environments. Consequently, performance of the example procedures is not limited to the example environment and the example environment is not limited to performance of the example procedures.

### Example Environment

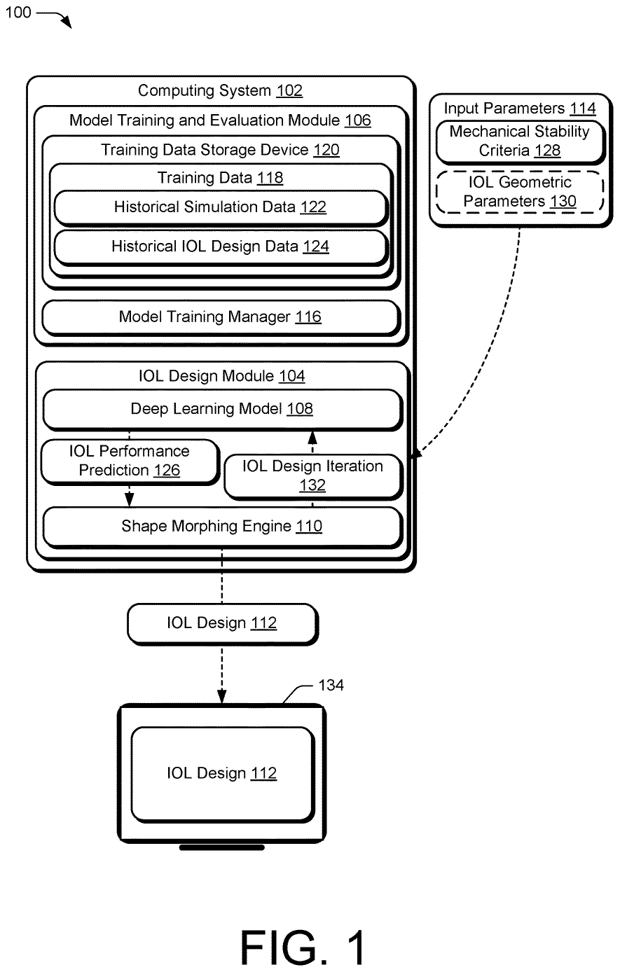

FIG. 1 is an illustration of an environment 100 in an example implementation that is operable to employ an intraocular lens (IOL) design generator for mechanical stability as described herein. The illustrated environment 100 includes a computing system 102 having an IOL design module 104 and a model training and evaluation module 106. The computing system 102 may be communicatively coupled to other devices via a network (not shown). The network may enable wired and/or wireless electronic communication, for example.

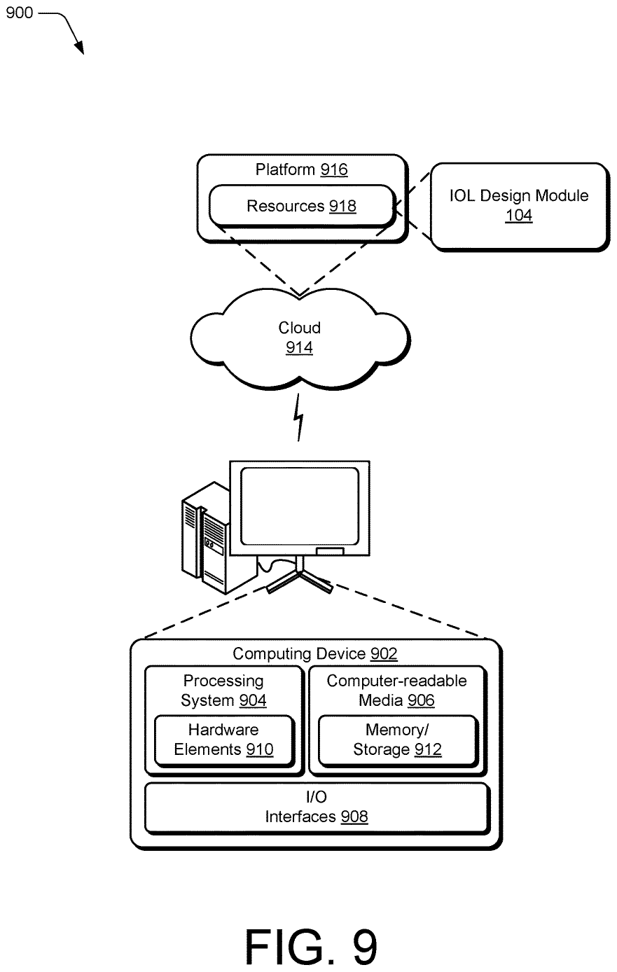

Computing devices that are usable to implement the computing system 102 may be configured in a variety of ways. A computing device, for instance, may be configured as a desktop computer, a laptop computer, a mobile device (e.g., assuming a handheld configuration such as a tablet or mobile phone), and so forth. Thus, the computing device may range from full resource devices with substantial memory and processor resources (e.g., personal computers, workstations) to a low-resource device with limited memory and/or processing resources (e.g., mobile devices). Additionally, a computing device may be representative of a plurality of different devices, such as multiple servers utilized to perform operations “over the cloud,” such as will be further described herein with reference to FIG. 9.

The IOL design module 104 includes a deep learning model 108 and a shape morphing engine 110 that work in conjunction to output an IOL design 112 based on input parameters 114, as will be elaborated herein. By way of example, the deep learning model 108 may provide performance predictions to the shape morphing engine 110, which may then iteratively modify an IOL design based on the input parameters 114 and the predictions. This interconnection between the deep learning model 108 and shape morphing engine 110 may enable rapid exploration of the IOL design space without performing time-consuming and resource-intensive computational physics-based simulations.

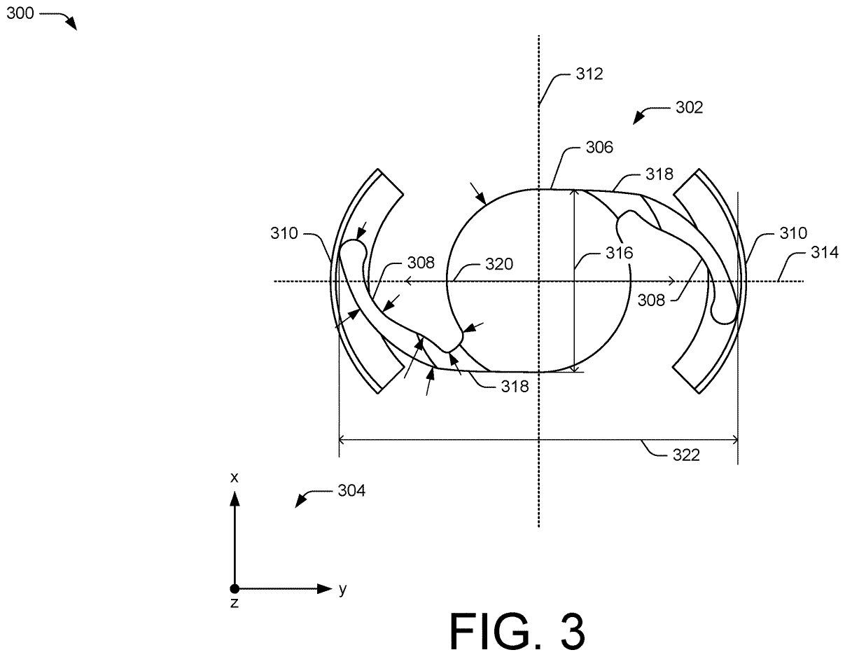

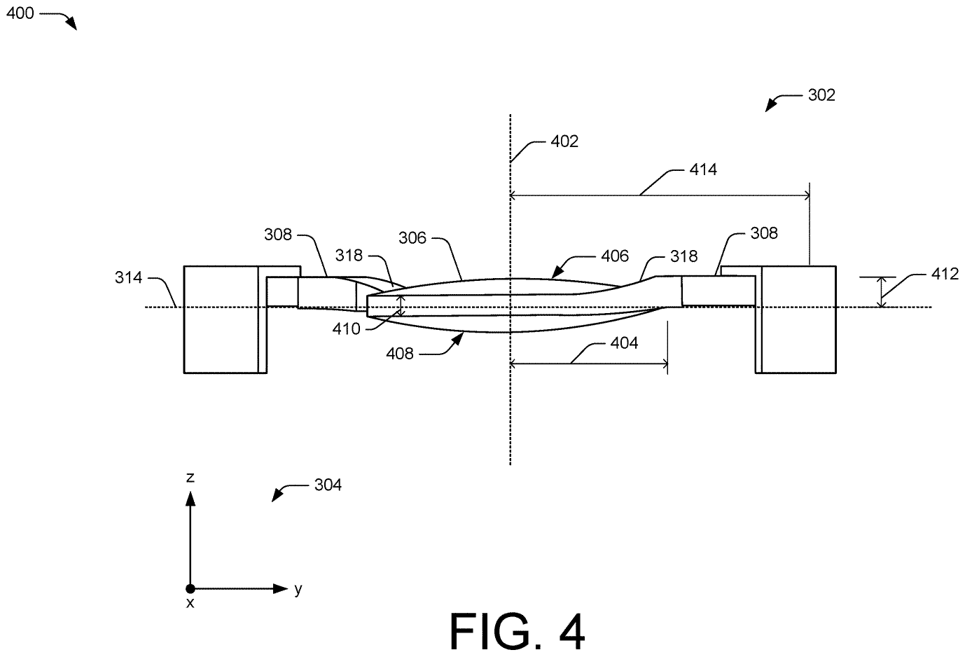

As used herein, an “IOL design” may include the geometric configuration, material properties, and/or structural characteristics of an intraocular lens. This may include, but is not limited to, parameters such as optic diameter (or radius), overall diameter (or radius), edge thickness, haptic angle, optic shape, haptic configuration, surface curvatures, refractive index, and material composition. Example parameters are given herein, e.g., with respect to FIGS. 3 and 4. An IOL design may also incorporate features related to mechanical stability, optical performance, biocompatibility, and/or manufacturability. In some implementations, an IOL design may be represented as a three-dimensional (3D) model, a set of parameters, or a combination of numerical and graphical data that collectively define the physical and functional attributes of the intraocular lens.

The act of “designing” may refer to the process of generating and refining intraocular lens configurations using deep learning techniques. This process may include analyzing input parameters, such as geometric specifications and/or mechanical stability criteria, to produce IOL designs that meet specified performance criteria. In this context, designing may include iteratively refining IOL geometries, predicting mechanical behavior, and exploring of the design space using the deep learning model 108 in combination with the shape morphing engine 110. The design process may encompass various stages, from initial design generation to finalization, and may leverage artificial intelligence algorithms to efficiently navigate complex relationships between IOL parameters and performance outcomes.

In at least one implementation, the model training and evaluation module 106 is implemented at least partially in hardware of the computing system to deploy deep learning to generate the deep learning model 108. By way of example, the model training and evaluation module 106 includes a model training manager 116 that is configured to manage the deep learning model 108. This model management may include, for example, building the deep learning model 108, training the deep learning model 108, updating the deep learning model 108, and so forth. For instance, the model training manager 116 may be configured to carry out this model management using, at least in part, training data 118 maintained in a training data storage device 120. The training data storage device 120 may include any known data storage medium.

It is to be appreciated that while the IOL design module 104, the model training and evaluation module 106, and the training data storage device 120 are illustrated as part of the computing system 102, in at least one variation, the IOL design module 104, the model training and evaluation module 106, and/or the training data storage device 120 is part of another computing device. Alternatively, or in addition, the components of the computing system 102 may be coupled to one another to form a single structure, may be separate but located within a common facility, or may be remotely located with respect to one another. For example, one or more of the devices and/or modules described herein may operate in a data server that has a distinct and remote location with respect to other components of the computing system 102. Optionally, the computing system 102 may be a unitary system that is capable of being accessed (e.g., remotely) from different locations. For example, the computing system 102 may include cloud computing capabilities, may be accessed via a secure network connection, or may be configured as a distributed computing environment.

In the environment 100 shown in FIG. 1, the training data 118 include historical simulation data 122 and historical IOL design data 124. The historical simulation data 122 may include data from previous physical tests, computational fluid dynamics models, and/or other computational simulations related to intraocular lens performance. The historical simulation data 122 may include results from mechanical stability tests, optical performance evaluations, and other relevant simulations that predict IOL behavior under various conditions. By way of example, the historical simulation data 122 may include information such as stress distributions, displacement values, optical aberrations, and other quantitative measures of IOL performance derived from computational models and/or physical tests. As non-limiting examples, the historical simulation data 122 may include mechanical properties and tests such as compression force, axial displacement in compression, optic decentralization, optic tilt, angle of contact, compression force decay, and dynamic fatigue durability.

The historical IOL design data 124 may include a database of previously designed and/or manufactured intraocular lens configurations that correspond to the historical simulation data 122. For instance, the historical IOL design data 124 may include geometric parameters of past IOL designs, such as optic diameter, overall diameter, edge thickness, haptic angle, various surface curvatures, jaws distance, neutral axis, haptic thickness or height, haptic ramp diameter, optic diameter anterior, optic diameter posterior, and various haptic surface radii. The historical IOL design data 124 may also contain information about the materials used in these designs, their optical properties, and any clinical or experimental performance data associated with the designs. This dataset may include both implemented (e.g., manufactured) and non-implemented design iterations from various sources such as manufacturers, research institutions, and clinical trials. In at least one implementation, the historical IOL design data 124 include human-generated IOL models, such as generated using computer-aided design (CAD) software.

In at least one implementation, entries in the historical simulation data 122 are paired with entries in the historical IOL design data 124 so that a given IOL design is associated with its corresponding historical simulation data 122. As will be further elaborated herein, the historical simulation data 122 may provide a ground-truth output for a given design of the historical IOL design data 124, which may facilitate training of the IOL design module 104. For example, the model training manager 116 may use at least a portion of the historical IOL design data 124 as input for training the deep learning model 108 and may use at least a portion of the historical simulation data 122 as feedback during training.

It is to be appreciated that although the historical simulation data 122 and historical IOL design data 124 are shown stored in the same training data storage device 120, in at least one variation, the historical simulation data 122 and historical IOL design data 124 are distributed among multiple storage locations. Alternatively, or in addition, the training data storage device 120 may be stored in a location that is external to the computing system 102 and accessed by the computing system 102 (e.g., over a network). As such, it is to be appreciated that the relative arrangement of the various modules and data storage devices in FIG. 1 is non-limiting, and variations are possible.

In one or more implementations, the training data 118 is further subdivided into a training sample, a development sample, and a validation sample. By way of example, the training sample may comprise a largest portion of the training data 118, while the validation sample may comprise a smallest portion of the training data 118. As a non-limiting example, the training sample comprises 70% of the training data 118, the development sample comprises 20% of the training data 118, and the validation sample comprises 10% of the training data 118, although other divisions are possible. The training sample, for instance, may comprise between 50% and 80% of the training data 118, the development sample may comprise between 10% and 40% of the training data 118, and the validation sample may comprise between 5% and 30% of the training data 118.

Broadly speaking, the training sample may be input to the deep learning model 108 during a training process, where the deep learning model 108 learns patterns and relationships in the data. By way of example, the deep learning model 108 may “learn” relationships between design parameters (e.g., as extracted from the historical IOL design data 124) and an outcome (e.g., mechanical stability performance, as extracted from the historical simulation data 122). During the training process, weights and parameters of the deep learning model 108 may be adjusted to reduce (e.g., minimize) errors between an output of the model and a ground truth label associated with a corresponding IOL design. Following completion of the training process, the deep learning model 108 may be able to output accurate IOL performance predictions 126 based on the training sample.

The development sample may be input to the deep learning model 108 during a model refinement (e.g., fine-tuning) process, where the deep learning model 108 is adjusted to prevent or reduce overfitting/underfitting of the model to the training sample. By way of example, the model refinement process may be performed following each round (or epoch) of training to evaluate how well the deep learning model 108 performs on data that is different from the training sample. During the model refinement process, for instance, a complexity, learning rate, and/or regularization of the deep learning model 108 may be adjusted (e.g., by the model training manager 116, automatically and/or based on user input) based on the performance of the deep learning model 108 with the development sample. As an illustrative example, if the deep learning model 108 accurately predicts the IOL performance predictions 126 of the training sample but not the development sample, overfitting of the deep learning model 108 to the training sample is indicated. As such, the model refinement process enables settings of the deep learning model 108 and/or its training to be fine-tuned so that the deep learning model 108 can be generalized to unseen data (e.g., data that the deep learning model 108 has not been trained on).

The validation sample may be input to the deep learning model 108 during a validation process that is performed after the deep learning model 108 is trained and fine-tuned. The validation sample comprises data that was unseen by the deep learning model 108 during the training and model refinement processes described above but that is derived from the same dataset (e.g., the training data 118). The validation process evaluates the performance of the deep learning model 108 on similar data as to that used during the training and model refinement processes. If the deep learning model 108 does not meet acceptable or desired performance criteria (e.g., as defined by model developers) during the validation process, the deep learning model 108 may be returned to the training and/or model refinement processes so that changes can be made. For example, changes may be made to feature selection, the model architecture, regularization techniques, hyperparameter tuning, and the like.

The model training manager 116 may leverage preprocessing functionality to process the training data 118 during the training, refinement, and validation processes described above. The preprocessing, for instance, may remove irrelevant information, standardize input formats, and/or normalize data. Additionally, or alternatively, the preprocessing may standardize IOL design data input into the deep learning model 108 in order to provide consistent inputs. The preprocessing, for instance, may include extracting values from CAD information, such as radius of curvature, optic diameter, haptic angle, overall diameter, edge thickness, and surface profiles. In some implementations, the preprocessing may also involve converting 3D mesh data into graph representations suitable for geometric deep learning algorithms.

In at least one implementation, the IOL design module 104 may include multiple different deep learning models that correspond to different types of machine learning models, where the underlying models learn using different approaches (e.g., supervised learning, unsupervised learning, and/or reinforcement learning), and/or multiple different deep learning models having a same model architecture but that are trained using different input data and/or to output different types of IOL performance predictions 126. By way of example, these models may include regression models (e.g., linear, polynomial, and/or logistic regression models), classifiers, neural networks, and reinforcement learning based models, to name just a few.

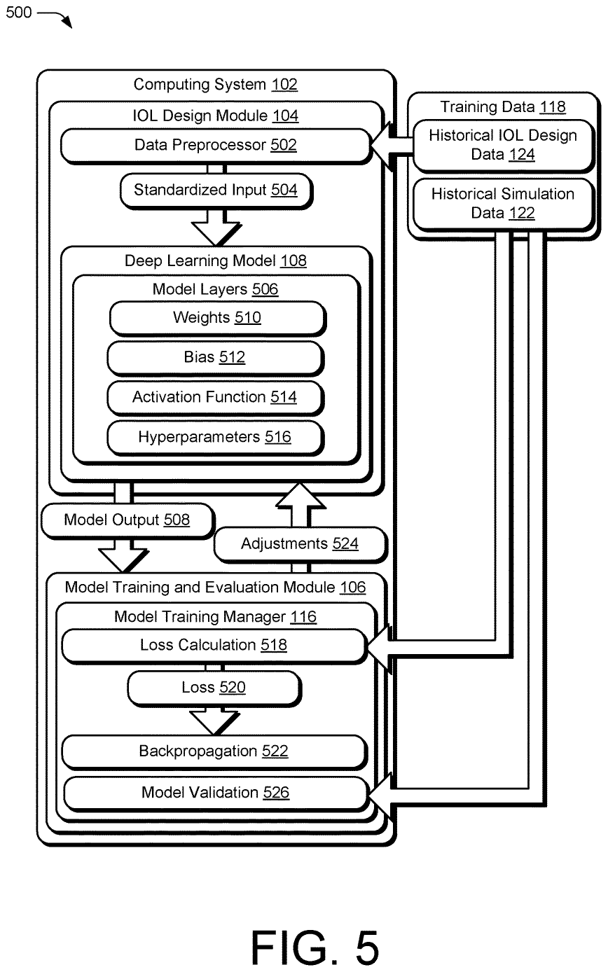

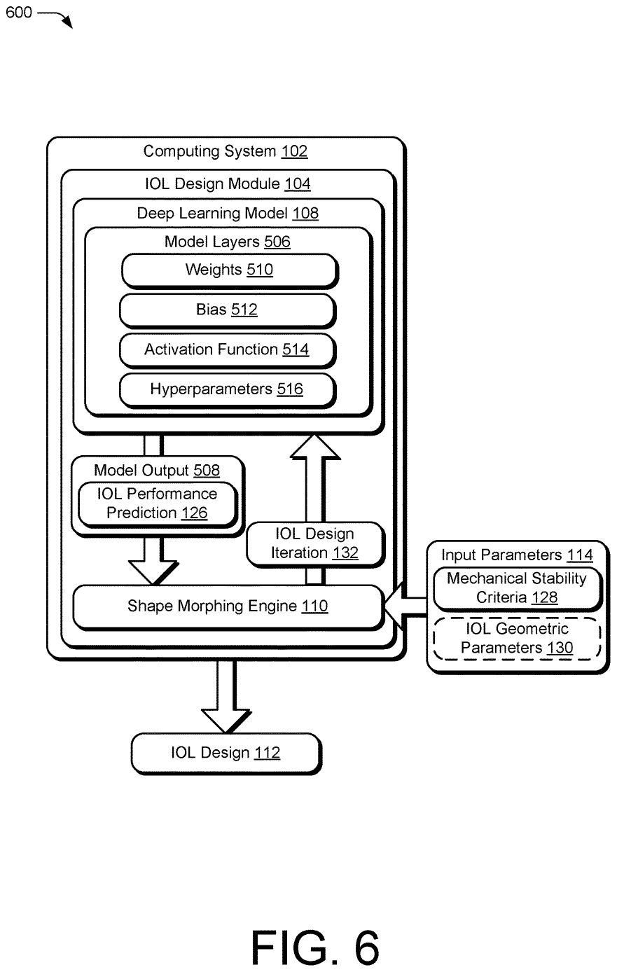

The deep learning model 108 may be configured as (or include) other types of models without departing from the spirit or scope of the described techniques. These different machine learning models may be built or trained (or the model otherwise learned), respectively, using different inputs and/or different algorithms due, at least in part, to different architectures and/or learning paradigms. Accordingly, it is to be appreciated that the following discussion of the functionality of the IOL design module 104 is applicable to a variety of machine learning models. For explanatory purposes, however, the functionality of the deep learning model 108 will be described generally with respect to a deep neural network. The deep neural network, for instance, may be a multi-layer neural network that includes three spatial dimensions to process 3D IOL geometries. By way of example, the deep learning model 108 may be based on a convolutional network architecture and/or a graph neural network architecture. Additional details of the deep neural network will be described herein, e.g., with respect to FIGS. 5 and 6. In one or more implementations, the deep neural network is combined with additional architectures and/or model portions to produce IOL performance predictions 126.

In response to the deep learning model 108 meeting desired or acceptable performance metrics following training, it may be deployed for generating the IOL performance predictions 126 for designing IOLs based on the input parameters 114, including parameters for which there is no pre-existing design. By way of example, the input parameters 114 may correspond, at least in part, to IOL design parameters and/or criteria that have not been previously explored.

In at least one implementation, the IOL design module 104 is configured to receive the input parameters 114. The input parameters 114 may include mechanical stability criteria 128, which may specify performance targets or constraints. The criteria 128, for instance, may specify a maximum allowable displacement under compression or desired stress distribution characteristics. In at least one implementation, optionally, the input parameters 114 further include IOL geometric parameters 130. The IOL geometric parameters 130 may include partial or high-level specifications, such as desired optic diameter range, overall diameter constraints, or a general haptic configuration. Alternatively, or in addition, the IOL geometric parameters 130 may include a previous IOL design (e.g., a baseline design) that is to be altered based on the mechanical stability criteria 128. The IOL design module 104 may use the input parameters 114 as constraints for generating the IOL design 112.

By way of example, the shape morphing engine 110 may receive the input parameters 114, including mechanical stability criteria 128 and optionally the IOL geometric parameters 130, as optimization objectives or constraints. The shape morphing engine 110 may use these to generate an IOL design iteration 132. In one or more implementations, the IOL design iteration 132 includes a three-dimensional (3D) IOL design. By way of example, the IOL design iteration 132 may define geometric parameters of the IOL (e.g., optic diameter, haptic angle, overall diameter, edge thickness). The deep learning model 108 may evaluate the IOL design iteration 132, predicting its mechanical stability performance and outputting its corresponding IOL performance prediction 126. The IOL performance prediction 126 may include an indication of the expected mechanical stability performance, including, e.g., a displacement under compression score. The IOL performance prediction 126 may be passed back to the shape morphing engine 110, which may compare the predicted performance to the desired criteria specified in the input parameters 114.

The IOL performance prediction 126 may be used by the shape morphing engine 110 to refine the IOL design iteration 132. By way of example, the shape morphing engine 110 may apply various topology modification algorithms and/or shape optimization techniques to modify the initial IOL design iteration 132, automatically and without user intervention, to generate an updated IOL design iteration 132. In some implementations, the shape morphing engine 110 employs topology modification algorithms such as mesh morphing based on control point displacement, free-form deformation using B-spline volumes, or gradient-based shape optimization in which the geometry is updated along a computed sensitivity of the predicted stability metric. During this iterative process, the shape morphing engine 110 may generate multiple IOL design iterations 132, with an objective to further improve on the expected mechanical stability performance. By way of example, the adjustments made by the shape morphing engine 110 may not be explicitly defined in the IOL geometric parameters 130, when included. The IOL design iteration 132 produced by the shape morphing engine 110 may be fed back to the deep learning model 108 to predict the expected mechanical stability performance of the IOL design iteration 132 (e.g., as the corresponding IOL performance prediction 126), and this process may be repeated until no additional mechanical stability gains are predicted. In at least one implementation, the IOL design module 104 may be configured to compare or rank the IOL design iterations 132 with respect to different criteria such as mechanical stability, optical performance, ease of manufacturing, and the like, and combinations thereof.

The best-ranked IOL design iteration 132, which may correspond to the IOL design iteration 132 having the highest mechanical stability according to the IOL performance prediction 126 in at least one implementation, is output as the (finalized) IOL design 112. This iterative approach may enable a dynamic and efficient exploration of the IOL design space, combining the predictive power of machine learning with the fine-tuning capabilities of shape optimization algorithms. By using the trained deep learning model 108 to rapidly predict mechanical stability, the IOL design module 104 may explore a wide range of potential designs generated by the shape morphing engine 110 without performing time-consuming physics-based simulations at each iteration. This significantly accelerates the IOL design process and may be used to generate IOL designs for a plurality of specific scenarios, including IOLs tailored to specific ophthalmic conditions, vision corrections, patient populations, and/or individual patients.

A display device 134 is shown displaying the IOL design 112. Alternatively, or in addition, the display device 134 may display the IOL performance prediction 126 and/or the IOL design iteration 132. In at least one implementation, the IOL performance prediction(s) 126, the IOL design iteration(s) 132, and/or the IOL design(s) 112 are stored in a memory of the computing system 102 in a manner to facilitate retrieval thereof, such as according to their order or time of generation.

In this way, the IOL design module 104 enables automated IOL design generation and optimization for mechanical stability, which may be used in rapid prototyping, design space exploration, and/or manufacturing process optimization. The IOL design module 104 may generate designs at a rate considerably faster than conventional methods, such as rates that are twenty times faster or more. However, it should be understood that the design generation rate may vary based on the complexity of the input parameters 114 and/or their similarity to the training data 118.

To facilitate the discussion of additional details of the intraocular lens design generator for mechanical stability, an overview of anatomic features of the eye will now be provided, followed by a discussion of an example intraocular lens.

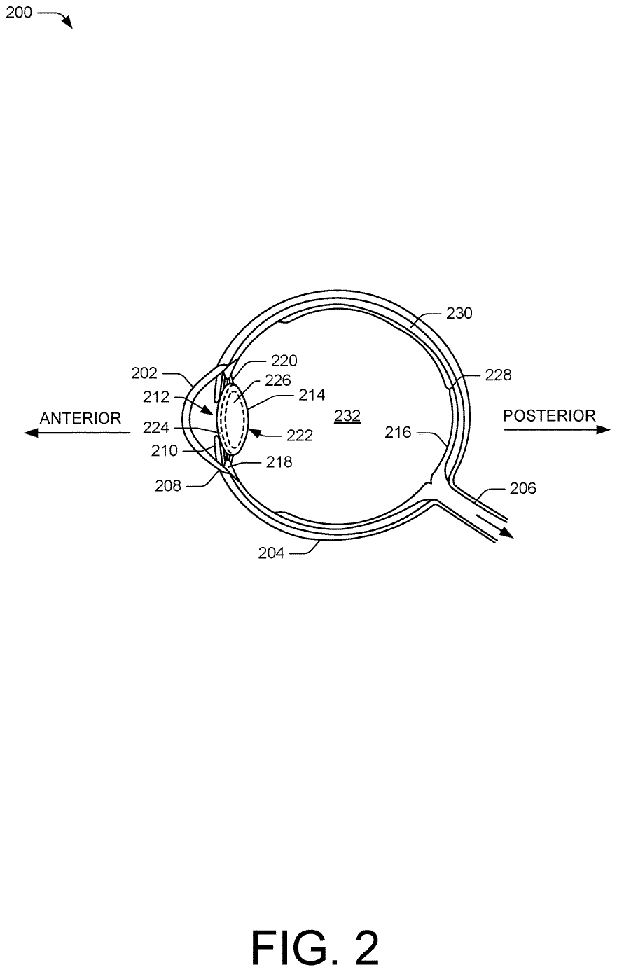

FIG. 2 depicts a cross-section of an eye 200. The eye 200 is a generally globular fused two-piece unit including a cornea 202 and sclera 204. The cornea 202 is a smaller transparent, dome-shaped front layer of the eye 200, whereas the sclera 204 is an opaque outer later that provides structural support and extends from the cornea 202 to an optic nerve 206. The cornea 202 is connected to the sclera 204 at a limbus 208. The limbus 208, for instance, is a ring that provides a transition zone between the cornea 202 and the sclera 204. The limbus 208 is typically about 11-12 millimeters (mm) in width (e.g., horizontal diameter) and 10-11 mm in height (e.g., vertical diameter), appearing elliptical in shape. The cornea 202 comprises approximately one-sixth of the outer surface area of the eye 200, whereas the sclera 204 comprises the remaining five-sixths. Moreover, the cornea 202 protrudes beyond the globe of the sclera 204 due to having a different (e.g., smaller) radius of curvature than the sclera 204.

The eye 200 further includes an iris 210. The iris 210 is the colored part of the eye 200 and surrounds a pupil 212. The iris 210 controls the size of the pupil 212, which is the central opening that allows light to enter the eye. The pupil 212 adjusts the size of the pupil 212 in response to light intensity, such as by contracting to reduce the size of the pupil 212 in response to bright light and dilating to increase the size of the pupil 212 in response to dim light. The iris 210 and the pupil 212 are seen instead of the cornea 202 due to the transparency of the cornea 202.

The eye 200 further includes a lens 214 (e.g., a crystalline lens) located behind the iris 210 and the pupil 212. The lens 214 is a transparent, flexible, biconvex structure that focuses light onto retina 216, which is a thin layer of tissue lining the back of the eye. The lens 214 is attached to ciliary body 218 by suspensory ciliary ligaments 220 (e.g., Zonules of Zinn), which include fine transparent fibers. The ciliary body 218 adjusts the lens shape for near or distant vision. By way of example, the lens 214, by changing its shape, functions to change the focal distance of the eye 200 so that it can focus on objects at various distances, thus allowing a sharp real image of the object of interest to be formed on the retina 216. This adjustment of the lens 214 is known as accommodation and is similar to the focusing of a photographic camera via movement of its lenses.

The lens 214 has three main parts: a lens capsule 222, a lens epithelium 224, and lens fibers 226. The lens capsule 222 (e.g., capsular bag) forms the outermost layer of the lens 214, and the lens fibers 226 form the bulk of the interior of the lens 214. The cells of the lens epithelium 224, located between the lens capsule 222 and the outermost layer of the lens fibers 226, are found predominantly on the anterior side of the lens but extend posteriorly just beyond the equator. The lens capsule 222 is a smooth, transparent basement membrane that completely surrounds the lens 214. The lens capsule 222 is elastic and thus causes the lens 214 to assume a more globular shape when not under the tension of the lens capsule 222. The lens capsule 222 varies between approximately 2-28 micrometers in thickness, being thickest near the equator and thinnest near the posterior pole.

The eye 200 is an organ that reacts to light for several purposes. By way of example, as a conscious sense organ, the eye allows vision. Rod and cone cells in the retina 216 allow conscious light perception and vision, including color differentiation and the perception of depth. In addition, non-image-forming photosensitive ganglion cells in the retina 216 receive light signals that affect adjustment of the size of the pupil 212, regulation and suppression of the hormone melatonin, and entrainment of the body clock.

The retina 216 includes macula 228, which is a small, central area that provides detailed central vision. The optic nerve 206 transmits visual information from the retina 216 to the brain, e.g., via electrical signals. A layer called choroid 230 between the retina 216 and the sclera 204 is a vascular layer that provides circulation to the retina 216.

Thus, the eye 200 is made up of three layers that enclose three transparent structures. The outermost layer includes the cornea 202 and sclera 204. The middle layer includes the choroid 230, the ciliary body 218, and the iris 210. The innermost layer is the retina 216, which gets its circulation from the vessels of the choroid 230 as well as the retinal vessels, which can be seen within an ophthalmoscope. Within these coats are aqueous humor, vitreous humor 232, and the lens 214. The aqueous humor is a clear fluid that is contained in two areas: an anterior chamber between the cornea 202 and the iris 210 and the exposed area of the lens 214; and the posterior chamber, between the iris 210 and the lens 214. The vitreous humor 232 is a clear, gel-like substance that fills the space between the lens 214 and the retina 216 that helps maintain the shape of the eye 200 and allows light to pass through to the retina 216.

The intraocular lens described herein is designed to replace the lens 214 of the eye 200 described in FIG. 2. When implanted, the IOL may be positioned within the lens capsule 222, which is the elastic membrane that normally encapsulates the natural lens. The IOL may interact with various anatomical structures of the eye, including the iris 210, pupil 212, and ciliary body 218. The shape and mechanical properties of the IOL may be optimized to work in conjunction with these structures to maintain proper positioning and provide optimal visual function. For example, the IOL may be designed to respond to the movement of the ciliary body 218, which typically adjusts the shape of the natural lens for accommodation. In at least one implementation, the size and curvature of the IOL may be tailored to fit within the available space of the eye 200, considering the dimensions of the cornea 202, anterior chamber, and posterior chamber, which may be given by the IOL geometric parameters 130 to develop patient-specific designs. Additionally, the IOL may be designed to focus light onto the retina 216, particularly the macula 228, to enable clear vision. The mechanical stability of the IOL, as determined by the IOL design module 104, may help ensure that the lens remains properly positioned within the eye 200 to provide consistent visual performance over time.

FIG. 3 depicts a top view 300 of an intraocular lens 302 with a bench test setup, and FIG. 4 depicts a side view 400 of the intraocular lens 302 with a bench test setup. Reference axes 304 in FIGS. 3 and 4 indicate a relative orientation of the top view 300 and the side view 400. By way of example, the reference axes 304 relate the views to a coordinate system having x, y, and z axes. The top view 300 of FIG. 3 is presented in the x-y plane, and the side view 400 is presented in the z-y plane. FIGS. 3 and 4 will be described collectively.

The intraocular lens 302 includes a central optical portion 306 and haptic portions 308 extending outward from the central optical portion 306. The haptic portions 308 are shown positioned in holders 310 in the present example, which may be used during manufacturing and/or testing (e.g., the bench test setup) but may not be implanted. The intraocular lens 302 may be further related to a midline 312 (shown in FIG. 3) that divides the intraocular lens 302 into left and right halves, an optical axis 314 (shown in FIGS. 3 and 4) that extends through the center of the central optical portion 306 and through the haptic portions 308 to divide the intraocular lens 302 into top and bottom portions, and central axis 402 (shown in FIG. 4), which denotes a plane of symmetry between the left and right halves. The midline 312 is parallel to the x-axis, the optical axis 314 is parallel to the y-axis, and the central axis 402 is parallel to the z-axis of the reference axes 304. The midline 312, the optical axis 314, and the central axis 402 intersect and are perpendicular to each other.

Various dimensions are also shown that define the design of the intraocular lens 302, at least in part. By way of example, the intraocular lens 302 has an optic diameter 316, which refers to the overall linear measurement of the central optical portion 306 across the midline 312. Additionally, or alternatively, an optic radius 404 may be used, which is shown extending from the central axis 402 in FIG. 4. As also shown in FIG. 4, the central optical portion 306 may be further defined by an anterior curvature profile 406 and a posterior curvature profile 408. In general, the anterior curvature profile 406 and the posterior curvature profile 408 indicate how steep or flat the corresponding surface of the central optical portion 306 is, which affects the focal length and optical power of the lens. The anterior curvature profile 406 may differ from the posterior curvature profile 408. In the present example, the central optical portion 306 has a bi-convex design, where the anterior (e.g., larger z-values with respect to the reference axes 304) and posterior (e.g., smaller z-values with respect to the reference axes 304) surfaces both curve outward with respect to the optical axis 314. In variations, the central optical portion 306 includes at least one concave surface that curves inward with respect to the optical axis 314. The central optical portion 306 further includes an edge thickness 410, which indicates the thickness at the periphery of the optic.

The haptic portions 308 may be connected to the central optical portion 306 via junctions 318, which may be adapted to give both flexibility and lens stability. The junctions 318, for instance, may be shaped to offset the haptic portions 308 from the optical axis 314. For example, as shown in FIG. 4, the haptic portions 308 are elevated (e.g., in the positive z-direction of the reference axes 304) relative to the optical axis 314. Each of the junctions 318 may include a sloped structure (e.g., a ramp) having a gradual incline, which may be referred to as an angle. Moreover, as shown in FIG. 3, a ramp diameter 320 may define the distance between the outermost points of the junctions 318 on opposite sides of the central optical portion 306.

The haptic portions 308 may further have a thickness 412, which may also be referred to as a height of the haptic portions 308. The thickness 412 may define a distance between the anterior and posterior surfaces of the haptic portions 308, such as when viewed in the side view 400 of FIG. 4. An overall diameter 322 may define an edge-to-edge maximum width (e.g., in the y-dimension of the reference axes 304) of the intraocular lens 302, from the outermost tip of one of the haptic portions 308 to the outermost tip of the opposite haptic portion 308. Alternatively, or in addition, this dimension may be defined as a radius 414, which measures a distance between the outermost tip of one of the haptic portions 308 and the central axis 402.

The design of the intraocular lens 302 may be further defined by additional or alternative widths, thicknesses, slopes, radii of curvature, and so forth. For instance, a plurality of different radii of curvature may be used to define surface curvature at specific locations of the intraocular lens 302, examples of which are indicated in FIG. 3 by single-headed arrows.

### Intraocular Lens Design Generator for Mechanical Stability

FIG. 5 depicts an example training process 500 that may be used in generating the deep learning model 108 of the IOL design module 104 of FIG. 1. It is to be appreciated that the example training process 500 denotes one implementation of a training process that may be used in generating at least a portion of the deep learning model 108, and variations are possible.

In the example training process 500, a training instance includes historical IOL design data 124 and historical simulation data 122 associated with the IOL design data. The historical IOL design data 124, for example, includes geometric parameters of past IOL designs, such as optic diameter, overall diameter, edge thickness, haptic angle, and surface curvatures. The historical IOL design data 124 may include designs with different configurations and parameters. Example configurations may include single-piece designs, multi-piece designs, various haptic designs, and various optic shapes. During the training, the historical IOL design data 124 is part of an input portion of the training data 118.

In at least one implementation, individual designs of the historical IOL design data 124 may be separately evaluated by the deep learning model 108 to generate a model output 508, which corresponds to the single IOL design. As such, each training instance may include an input portion (e.g., the historical IOL design data 124) and an associated expected output portion (e.g., the historical simulation data 122), and a great many training instances may be used during the training process. The model output 508 corresponds to a prediction for IOL performance characteristics (e.g., the IOL performance prediction 126 for the corresponding historical IOL design data 124) that can be compared to the corresponding historical simulation data 122. By way of example, the deep learning model 108 may be trained to output (e.g., as the model output 508) mechanical stability predictions, optical performance predictions, and/or combinations thereof. The historical simulation data 122 defines a true or expected output of the deep learning model 108 for the input IOL design data.

In the example shown in FIG. 5, the historical IOL design data 124 is input to a data preprocessor 502, which generates a standardized input 504. The data preprocessor 502 may extract relevant geometric features and/or values (e.g., measurements), normalize pixel values, and/or convert 3D mesh data into representations suitable for processing by the deep learning model 108. By way of example, different IOL designs may have different representations. The standardization process performed by the data preprocessor 502 may ensure a consistent input to the deep learning model 108.

The standardized input 504 is fed into the deep learning model 108. In the example training process 500 shown in FIG. 5, the deep learning model 108 may include a deep neural network, such as a convolutional neural network (CNN) or graph neural network (GNN), having multiple model layers 506. These layers are configured to process the 3D geometric information of the IOL design. The model layers 506 may include multiple layers that progressively extract and process features from the input data. For instance, earlier layers may detect basic geometric features like edges and curves, while later layers may identify more complex patterns such as an overall lens shape and/or haptic configuration. In at least some implementations, the model layers 506, or a portion thereof, may be arranged in blocks, with each block capable of extracting increasingly abstract features of the input IOL design. As a non-limiting example, the model layers 506 include five layers each having a plurality of neurons.

Each of the model layers 506 may include weights 510, a bias 512, an activation function 514, and hyperparameters 516. The weights 510 and bias 512 may be randomly initialized and then “learned” during the training process. The model layers 506 perform a series of operations on the input data, which may involve message passing between nodes in the graph representation of the IOL design. After each operation, the result may be passed through the activation function 514, e.g., a non-linear function such as ReLU (Rectified Linear Unit), a Softmax function, or another type of activation function selected according to a task of the given model layer.

The hyperparameters 516 are not learned during the training process but can be adjusted to increase performance. These may include parameters such as the number of layers, the size of the hidden layers, and the learning rate. Adjusting the hyperparameters 516 can affect the ability of the deep learning model 108 to relate complex geometric relationships in the IOL designs to performance metrics and impact the computational efficiency of the training process.

The deep learning model 108 is designed to process 3D IOL models (e.g., human-generated CAD files), allowing the deep learning model 108 to capture geometric relationships and structural features that are tied to predictions of mechanical stability and/or optical performance. This approach enables the model to “learn” from the complex, three-dimensional nature of IOL designs.

In at least one implementation, convolutional layers of the model layers 506 may output a design embedding, which may be a flattened representation of geometric features of the IOL design. The design embedding may be a one-dimensional vector that summarizes the features or characteristics of the standardized input 504 that have been learned by the deep learning model 108. The design embedding may be input into one or more dense layers, which are fully connected layers where every neuron (node) is fully connected to every neuron in the previous and next layer. The one or more dense layers function to aggregate information learned by previous layers and make final predictions for the model output 508.

Similar to the layers described above, the one or more dense layers may comprise the weights 510, the bias 512, the activation function 514, and the hyperparameters 516. It is to be appreciated that values of at least a portion of these parameters and/or type of activation function used are specific to a given model layer 506 or dense layer. The one or more dense layers, for instance, may take the design embedding as an input vector, multiply it by a matrix of the weights 510, add the bias 512, and then apply the activation function 514 to produce the model output 508.

The model output 508 is received by the model training manager 116, which may perform a loss calculation 518. The loss calculation 518 may use a loss function to compute the difference between the model output 508 (predicted values) and the historical simulation data 122 (true values), e.g., a loss 520. The loss 520, for instance, is a measure of the error of the deep learning model 108 in determining the model output 508. The loss function used in the loss calculation 518 may depend on a type of the model output 508. As non-limiting examples, mean squared error loss may be used for regression problems (where a continuous value is predicted), whereas cross-entropy loss may be used for classification problems.

A goal of the training is to minimize the loss 520 by adjusting the weights 510 and biases 512 of the deep learning model 108. In order to do so, the model training manager 116 may employ backpropagation 522 to compute how the parameters are to be updated based on a gradient of the loss 520 with respect to each parameter. By way of example, the backpropagation 522 process may utilize an optimization algorithm, such as gradient descent. The backpropagation 522 results in adjustments 524, which are used to update the weights 510 and biases 512 of the deep learning model 108.

As such, following many rounds of training with a large number of training instances, the model output 508 becomes consistent with the historical simulation data 122 due to the deep learning model 108 “learning” to minimize the loss 520 between the model output 508 and the historical simulation data 122.

Following the training, the model training manager 116 may perform model validation 526 prior to deployment of the deep learning model 108 to verify its ability to accurately predict the IOL performance of IOL designs that are not used to train the model. The model validation 526 includes comparing a predicted performance (e.g., the model output 508) to a true performance (e.g., the historical simulation data 122) for a validation dataset portion of the training data 118. The model validation 526 is not used to apply the adjustments 524 to the weights 510 and biases 512. Rather, the model validation 526 evaluates a performance of the deep learning model 108. If the deep learning model 108 does not meet acceptable or desired performance criteria during the model validation 526, the deep learning model 108 may be retrained and/or refined. For example, changes may be made to feature selection, the model architecture, regularization techniques, hyperparameter tuning, and the like.

The training process 500 thus provides a general framework for further model adaptation to generate specific IOL performance prediction 126 outputs. By way of example, the model layers 506 may provide an encoder backbone that can be combined with additional architectures to produce the IOL performance prediction 126. Moreover, by using this example training process 500, the deep learning model 108 does not rely on manual design annotation but instead “learns” which geometric features of the IOL design are relevant to a given performance criterion. This also enables the deep learning model 108 to identify data patterns in IOL designs and corresponding performance data beyond what is interpretable by a human in manual design workflows. For instance, the deep learning model 108 may be trained to output a prediction regarding one or more specific mechanical stability characteristics of the IOL (e.g., deformation at the IOL center). However, the prediction output by the deep learning model 108 is an estimation, as the deep learning model 108 does not perform a physical simulation on the input data. Because the deep learning model 108 does not perform a physical simulation, the deep learning model 108 may be able to estimate the performance in part using latent information from design features.

FIG. 6 depicts an example implementation 600 of using the deep learning model 108 to design an intraocular lens based on desired mechanical stability criteria. Components previously introduced in the preceding figures are numbered the same and function as previously described.

In the implementation 600, the IOL design module 104 receives the input parameters 114, including the mechanical stability criteria 128 and optionally the IOL geometric parameters 130. The shape morphing engine 110 may use these input parameters as optimization objectives or constraints to generate the (initial) IOL design iteration 132, which may be represented as a 3D model and/or parametric descriptions. The IOL design iteration 132 is then passed to the deep learning model 108 for evaluation.

The deep learning model 108 is trained to output, as the model output 508 from the model layers 506, the IOL performance prediction 126. The IOL performance prediction 126, for instance, may indicate an expected mechanical stability performance, which may provide quantitative measure estimates for the input IOL design iteration 132. The IOL performance prediction 126 may further include predictions for optical performance. By way of example, the mechanical stability performance predictions may correspond to how well the IOL is expected to maintain its position under various forces (e.g., displacement under compression). In general, higher stability corresponds to better performance of the IOL. A low stability score, for instance, may indicate a higher risk of IOL displacement. As such, the mechanical stability performance predictions may provide one indication of the function of the IOL specified by the geometric design parameters.

The optical performance predictions may include one or both of the refractive power and aberration characteristics. The refractive power corresponds to the IOL's ability to focus light, while the aberration characteristics correspond to the quality of the focused image. Better optical performance predictions may indicate that the IOL design iteration 132 is expected to be more effective at correcting vision. As such, the optical performance metrics may provide another indication of the expected function of the IOL of the IOL design iteration 132.

It is to be appreciated that the IOL measurement predictions may include one or more additional or alternative measurements of IOL performance and/or function from those explicitly listed without departing from the spirit or scope of the described techniques.

In at least one implementation, the shape morphing engine 110 may receive the IOL performance prediction 126 from the deep learning model 108 and may employ iterative algorithms to gradually modify the IOL design. By way of example, the shape morphing engine 110 may adjust one or more IOL design parameters and/or alter the overall shape or configuration of the IOL in an iterative fashion. The IOL design parameters may include one or more diameters and/or radii, the haptic configuration (including, for example, thickness and height), a plurality of different curvatures, and so forth, such as those described above with respect to FIGS. 3 and 4. An optic diameter, for instance, may refer to the size of the central optical zone of the IOL, whereas the haptic configuration corresponds to the design of the supporting structures that hold the IOL in place. Changes in these parameters may affect both the mechanical stability and optical performance of the IOL, as determined by processing the (updated) IOL design iteration 132 with the deep learning model 108. By way of example, the geometric design parameters directly relate to the mechanical stability predictions and/or the optical performance predictions according to relationships learned by the deep learning model 108. At least a portion of these relationships may be “latent” relationships that are not readily apparent to a human user. For instance, a human user may not be able to predict the effect of changing a surface curvature on the mechanical stability of the IOL.

Accordingly, the deep learning model 108 may estimate the performance of the modified design for each IOL design iteration 132, thus avoiding time-consuming computational physics-based simulations. The shape morphing engine 110 may generate multiple IOL design iterations 132, exploring different regions of the design space.

In at least one implementation, the shape morphing engine 110 may continue modifying the IOL design via the IOL design iterations 132 until predetermined performance thresholds are met or no further mechanical stability gains are predicted. By way of example, the shape morphing engine 110 may introduce geometric features not explicitly defined in the IOL geometric parameters 130 but that do not directly conflict with the IOL geometric parameters 130, when provided. This iterative process enables a rapid exploration of the IOL design space, and the deep learning model 108 enables the IOL design module 104 to efficiently navigate complex relationships between IOL parameters and performance outcomes.

It is to be understood that as used herein, an “IOL design iteration” corresponds to one or more modified designs generated by the shape morphing engine 110 and is not meant to limit the iterations to a single output.

Through the interconnection of the deep learning model 108 and the shape morphing engine 110, the IOL design module 104 arrives at an overall refined and improved (e.g., with respect to mechanical stability) IOL design that meets the specified input parameters 114, which is output as the IOL design 112.

Thus, the implementation 600 shown in FIG. 6 may allow the IOL design module 104 to process multiple designs and iterations of IOL data to generate comprehensive IOL structure and function predictions from the input parameters 114, which may be tailored to a specific patient's needs or generalized for a specific vision correction. By linking the deep learning model 108 and the shape morphing engine 110, the IOL design module 104 may significantly reduce the time and resources used for IOL design iterations. The ability of the deep learning model 108 to generate comprehensive predictions of IOL structure and function may enhance the efficiency of the design process. Furthermore, the ability of the deep learning model 108 to estimate IOL performance without conducting physical simulations may accelerate design evaluation. Accordingly, the IOL design module 104 may facilitate the development of more stable and effective IOLs, which may improve surgical outcomes and patient satisfaction in cataract and refractive procedures.

Having discussed example details of the techniques for an intraocular lens design generator for mechanical stability, consider now example procedures to illustrate additional aspects of the techniques.

### Example Procedures

This section describes example procedures for an intraocular lens design generator for mechanical stability in one or more implementations. Aspects of the procedures may be implemented in hardware, firmware, or software, or a combination thereof. The procedures are shown as a set of blocks that specify operations performed by one or more devices and are not necessarily limited to the orders shown for performing the operations by the respective blocks. In at least some implementations, at least a portion of the procedures are performed by a suitably configured device, such as the computing system 102 of FIG. 1, by executing instructions stored in a non-transitory computer-readable storage medium.

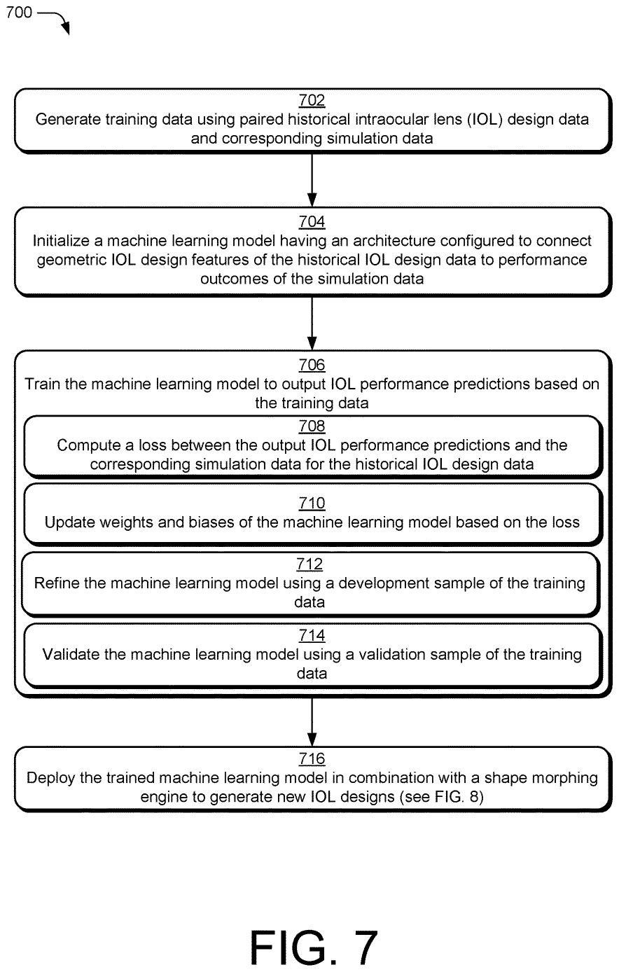

FIG. 7 depicts an example procedure 700 in which a deep learning model is trained to predict intraocular lens device performance based on geometry.

Training data is generated using paired historical IOL design data and corresponding simulation data (block 702). By way of example, the training data 118 may include the historical IOL design data 124 and the historical simulation data 122, which may be stored in the training data storage device 120. The historical IOL design data 124 may include geometric parameters of past IOL designs, such as optic diameter, overall diameter, edge thickness, haptic angle, and surface curvatures. The historical simulation data 122 may include results from mechanical stability tests, optical performance evaluations, and/or other relevant simulations and/or physical tests that predict IOL behavior under various conditions. In at least one implementation, the training data 118 is subdivided into a training sample (e.g., 70% of the data), a development sample (e.g., 20% of the data), and a validation sample (e.g., 10% of the data).

A machine learning model is initialized with an architecture configured to connect geometric IOL design features of the historical IOL design data to performance outcomes of the simulation data (block 704). By way of example, the deep learning model 108 may include with model layers 506 designed to process 3D IOL models and output performance predictions. The model architecture may include convolutional layers for processing 3D geometric information and dense layers for final predictions, for example. Initializing the machine learning model may include setting initial values for weights 510, biases 512, and hyperparameters 516. The weights 510 and biases 512 may be randomly initialized within certain ranges. The hyperparameters 516, such as learning rate, number of layers, and size of hidden layers, may be set (e.g., by a developer of the machine learning model) based on empirical testing or domain knowledge. In some implementations, the activation functions 514 for each layer may also be specified during initialization. This initialization process may provide a starting point for the model before training begins, allowing it to learn the complex relationships between IOL geometry and performance during the subsequent training.

The machine learning model is trained to output IOL performance predictions based on the training data (block 706). In accordance with the techniques described herein, training the machine learning model may include computing a loss between the output IOL performance predictions and the corresponding simulation data for the historical IOL design data (block 708). By way of example, the model training manager 116 may perform a loss calculation 518 to compute the difference between the model output 508 and the historical simulation data 122 for a corresponding input historical IOL design data 124. The loss function used may depend on the type of prediction, such as mean squared error for regression problems or cross-entropy loss for classification problems.

Training the machine learning model may further include updating weights and biases of the machine learning model based on the computed loss (block 710). By way of example, the model training manager 116 may employ backpropagation 522 to compute how the weights 510 and biases 512 are to be updated based on a gradient of the loss 520. This process may include adjusting the weights 510 and biases 512 of the model layers 506 to minimize the loss.

In one or more implementations, the overall training process may include distinct phases using different subsets of the data to ensure robust model performance. The training sample, which may comprise the largest portion of the training data 118, may be used during a model training phase. During this phase, the machine learning model “learns” to map input features to output predictions by adjusting its internal parameters, such as mentioned above.

Accordingly, training the machine learning model may further include refining the machine learning model using a development sample of the training data (block 712). The development sample, which may comprise a smaller portion of the data, may be used during a model refinement phase. The development sample may help prevent overfitting by providing a set of data that the model has not directly trained on. The model's performance on the development sample may guide adjustments to the model architecture, learning rate, or other hyperparameters 516.

Training the machine learning model may further include validating the machine learning model using a validation sample of the training data (block 714). The validation sample, which may comprise the smallest portion of the data in some examples, may be used during a validation phase of training the machine learning model. The validation sample may provide an unbiased assessment of the model's performance on unseen data, simulating real-world application. The model's performance on the validation sample may indicate its generalization capability and readiness for deployment. The model validation 526, for example, may evaluate the performance of the machine learning model by comparing a predicted performance (e.g., the IOL performance prediction 126 output by the deep learning model 108) to a true performance (e.g., the historical simulation data 122). If the machine learning model does not meet acceptable or desired performance criteria (e.g., as defined by model developers) during the model validation 526, the machine learning model may be returned to the training and/or model refinement processes described above so that changes can be made. For example, changes may be made to feature selection, the model architecture, regularization techniques, hyperparameter tuning, and the like.

By utilizing these distinct data subsets, the training process may aim to produce a model that not only performs well on the training data, but also generalizes effectively to new, unseen IOL designs. This approach may help ensure the reliability and robustness of the machine learning model in generating accurate IOL performance predictions.

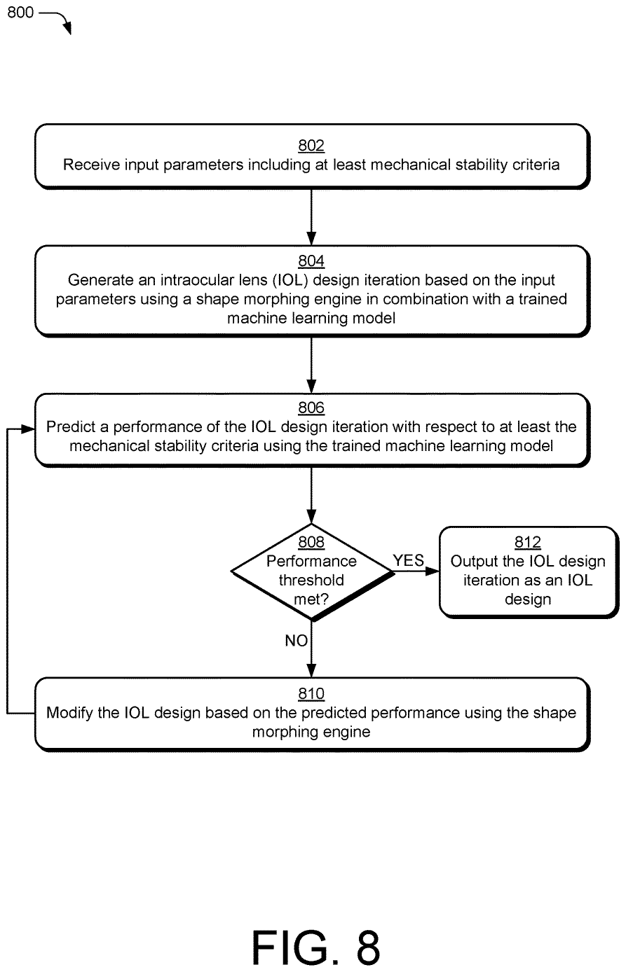

The trained machine learning model is deployed in combination with a shape morphing engine to generate new IOL designs (block 716). By way of example, the deep learning model 108 may be combined with the shape morphing engine 110 in the IOL design module 104 to generate new IOL designs based on input parameters 114, such as will be described with respect to FIG. 8. Accordingly, the trained machine learning model may be used to process multiple designs and iterations of IOL data to generate comprehensive IOL structure and function predictions.

FIG. 8 depicts an example procedure 800 in which new intraocular lens (IOL) designs are generated using a deep learning model.

Input parameters including at least mechanical stability criteria are received (block 802). By way of example, the input parameters 114 may include mechanical stability criteria 128 and optionally IOL geometric parameters 130. The mechanical stability criteria 128 may specify performance targets or constraints, such as maximum allowable displacement under compression and/or desired stress distribution characteristics. The IOL geometric parameters 130 may include a baseline IOL design and/or partial or high-level specifications (e.g., a desired optic diameter range, overall diameter constraints, and/or a general haptic configuration). The input parameters 114 may be tailored to a specific patient, generalized for a specific vision correction, and/or for specific properties (e.g., thickness, elasticity, etc.) of a material from which the IOL will be manufactured.

An IOL design iteration is generated based on the input parameters using a shape morphing engine in combination with a trained machine learning model (block 804). By way of example, the shape morphing engine 110 may use the input parameters 114 as optimization objectives or constraints for generating the IOL design iteration 132, which may be represented as a 3D model and/or parametric descriptions. The shape morphing engine 110 may apply various shape optimization techniques to modify the initial design. This may include adjusting parameters such as optic diameter, haptic angle, overall diameter, edge thickness, and/or surface curvatures. The shape morphing engine 110 may also introduce geometric features not explicitly defined in the IOL geometric parameters 130 but that do not directly conflict with the IOL geometric parameters 130.

A performance of the IOL design iteration is predicted with respect to the mechanical stability criteria using the trained machine learning model (block 806). By way of example, the deep learning model 108 may receive the IOL design iteration 132 and generate the corresponding IOL performance prediction 126. The IOL performance prediction 126 may include an indication of the expected mechanical stability performance (e.g., a displacement under compression score or a probability score indicating the likelihood that the design meets specified mechanical stability criteria). The IOL performance prediction 126 may further include predictions for optical performance, at least in some implementations.

By way of example, the deep learning model 108, may be based on a convolutional network architecture and/or a graph neural network architecture and may process the 3D geometric information of the IOL design iteration 132. The model layers 506 may include multiple layers that progressively extract and information. For instance, earlier layers may detect basic geometric features like edges and curves, while later layers may identify more complex patterns such as an overall lens shape and/or haptic configuration.

The deep learning model 108 may further include layers that output predictions based on the extracted information in order to estimate the mechanical stability performance of the IOL design iteration 132 by leveraging patterns and relationships learned from the training data 118, which include historical simulation data 122 and historical IOL design data 124. This allows the model to generalize its predictions to new, unseen IOL designs, enabling efficient evaluation of novel configurations without performing time-consuming and resource-intensive computational physics-based simulations. This approach enables rapid and automated exploration of the IOL design space, as further described herein.

It is determined whether a performance threshold has been met (block 808). By way of example, the IOL design module 104 may compare the predicted performance from the IOL design prediction 126 to the specified mechanical stability criteria 128. This comparison may involve evaluating multiple performance metrics simultaneously. In at least one implementation, the performance threshold may be a composite score or a set of individual thresholds for different criteria. Alternatively, or in addition, the performance threshold may specify a maximum number of iterations to perform and/or a desired improvement factor (e.g., a percentage value). As a non-limiting, illustrative example, the performance factor may specify an improvement factor of at least 30% with respect to IOL deformation at the center compared to a baseline measurement.

In response to the performance threshold not being met (e.g., “NO”), the IOL design is modified based on the predicted performance using the shape morphing engine (block 810). By way of example, the shape morphing engine 110 may generate multiple IOL design iterations 132, with an objective to further improve on the IOL performance prediction 126. The shape morphing engine 110 may adjust one or more IOL design parameters and/or alter the overall shape or configuration of the IOL in an iterative fashion. These adjustments may be guided by the IOL performance prediction 126 from the deep learning model 108, allowing for efficient exploration of the design space. The procedure 800 may return to block 806 to predict the performance of the modified design, creating an iterative loop until the performance threshold is met at block 808.

In response to the performance threshold being met (e.g., “YES”), the IOL design iteration is output as an IOL design (block 812). By way of example, the IOL design module 104 may output the final IOL design 112, which meets the specified input parameters 114 and the performance threshold. The output may include a 3D model of the IOL and/or parametric descriptions of its geometry. In at least some implementations, the output further includes the corresponding IOL performance prediction 126. The IOL design 112 may be further analyzed (e.g., using physical test and/or physics-based computations), prototyped, and/or manufactured for use in patients.

By generating and using a trained machine learning model for IOL design outcome predictions, more stable and effective IOLs may be designed while a development time and computing resource usage is reduced. As a non-limiting, illustrative example, the trained machine learning model may output the IOL performance prediction 126 in approximately twenty seconds. In contrast, a conventional physics-based computational approach may take approximately seven minutes to output a similar IOL performance prediction. By quickly exploring the IOL design space using the combination of the deep learning model 108 and the shape morphing engine 110, surgical outcomes and patient satisfaction in IOL implantation procedures may be improved.

Having described example procedures in accordance with one or more implementations, consider now an example system and device that can be utilized to implement the various techniques described herein.

### Example System and Device

FIG. 9 illustrates an example system generally at 900 that includes an example computing device 902 that is representative of one or more computing systems and/or devices that may implement the various techniques described herein. The computing device 902 may be, for example, a server of a service provider, a device associated with a client (e.g., a client device), an on-chip system, and/or any other suitable computing device or computing system. The computing device 902 is one example of the computing system 102 of FIG. 1, for example.

The example computing device 902 as illustrated includes a processing system 904, one or more computer-readable media 906, and one or more I/O interfaces 908 that are communicatively coupled, one to another. Although not shown, the computing device 902 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 904 is representative of functionality to perform one or more operations using hardware. Accordingly, the processing system 904 is illustrated as including hardware elements 910 that may be configured as processors, functional blocks, and so forth. This may include implementation in hardware as an application specific integrated circuit or other logic device formed using one or more semiconductors. The hardware elements 910 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically executable instructions.

The computer-readable storage media 906 is illustrated as including memory/storage 912. The memory/storage 912 represents memory/storage capacity associated with one or more computer-readable media. The memory/storage 912 may include volatile media (such as random-access memory (RAM)) and/or nonvolatile media (such as read only memory (ROM), Flash memory, optical disks, magnetic disks, and so forth). The memory/storage 912 may include fixed media (e.g., RAM, ROM, a fixed hard drive, and so on) as well as removable media (e.g., flash memory, a removable hard drive, an optical disc, and so forth). The computer-readable media 906 may be configured in a variety of other ways as further described below.

Input/output interface(s) 908 are representative of functionality to allow a user to enter commands and information to computing device 902, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 902 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

For instance, the terms “module,” “functionality,” and “component” may include a hardware and/or software system that operates to perform one or more functions. For example, a module, functionality, or component may include a computer processor, a controller, or another logic-based device that performs operations based on instructions stored on a tangible and non-transitory computer-readable storage medium, such as a computer memory. Alternatively, a module, functionality, or component may include a hardwired device that performs operations based on hardwired logic of the device. Various modules, systems, and components shown in the attached figures may represent the hardware that operates based on software or hardwired instructions, the software that directs hardware to perform the operations, or a combination thereof.

An implementation of the described modules and techniques may be stored on or transmitted across some form of computer-readable media. The computer-readable media may include a variety of media that may be accessed by the computing device 902. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media, and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 902, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 910 and computer-readable media 906 are representative of modules, programmable device logic and/or fixed device logic implemented in a hardware form that may be employed in some examples to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 910. The computing device 902 may be configured to implement particular instructions and/or functions corresponding to the software and/or hardware modules. Accordingly, implementation of a module that is executable by the computing device 902 as software may be achieved at least partially in hardware, e.g., through use of computer-readable storage media and/or hardware elements 910 of the processing system 904. The instructions and/or functions may be executable/operable by one or more articles of manufacture (for example, one or more computing devices 902 and/or processing systems 904) to implement techniques, modules, and examples described herein.

The techniques described herein may be supported by various configurations of the computing device 902 and are not limited to the specific examples of the techniques described herein. This functionality may also be implemented all or in part through use of a distributed system, such as over a “cloud” 914 via a platform 916 as described below.

The cloud 914 includes and/or is representative of a platform 916 for resources 918, which are depicted including the IOL design module 104 in the present example. It is to be appreciated, however, that in other implementations, the IOL design module 104 may run locally on a client server or computer rather than on the platform 916. The platform 916 abstracts underlying functionality of hardware (e.g., servers) and software resources of the cloud 914. The resources 918 may include applications and/or data that can be utilized while computer processing is executed on servers that are remote from the computing device 902. Resources 918 can also include services provided over the Internet and/or through a subscriber network, such as a cellular or Wi-Fi network.

The platform 916 may abstract resources and functions to connect the computing device 902 with other computing devices. The platform 916 may also serve to abstract scaling of resources to provide a corresponding level of scale to encountered demand for the resources 918 that are implemented via the platform 916. Accordingly, in an interconnected device example, implementation of functionality described herein may be distributed throughout the system 900. For example, the functionality may be implemented in part on the computing device 902 as well as via the platform 916 that abstracts the functionality of the cloud 914.

### Conclusion

Although the invention has been described in language specific to structural features and/or methodological acts, it is to be understood that the invention defined in the appended claims is not necessarily limited to the specific features or acts described. Rather, the specific features and acts are disclosed as example forms of implementing the claimed invention.

What is claimed is:

1. A system for generating an intraocular lens (IOL) design, comprising:
an IOL design module implemented in a non-transitory computer-readable storage medium and configured to perform operations comprising:
generate an IOL design iteration based at least on input mechanical stability criteria using a shape morphing engine;
predict a performance of the IOL design iteration with respect to the input mechanical stability criteria using a machine learning model trained on historical IOL design data and corresponding historical simulation data; and
output an IOL design based on the predicted performance of the IOL design iteration.

2. The system of claim 1, wherein the IOL design iteration is further based on IOL geometric parameters comprising at least one of an optic diameter, an overall diameter, an edge thickness, a haptic angle, a surface curvature, or a baseline IOL design.

3. The system of claim 1, wherein predicting the performance of the IOL design iteration comprises:
extracting three-dimensional geometric information of the IOL design iteration using a deep neural network of the machine learning model; and
estimating a mechanical stability of the IOL design iteration based on the extracted three-dimensional geometric information using the machine learning model.

4. The system of claim 1, wherein outputting the IOL design based on the predicted performance of the IOL design iteration comprises:
determining whether a performance threshold has been met based on the predicted performance; and
in response to the performance threshold being met, outputting the IOL design iteration as the IOL design; or
in response to the performance threshold not being met, adjusting the IOL design iteration via the shape morphing engine.

5. The system of claim 4, wherein adjusting the IOL design iteration comprises:
modifying, via the shape morphing engine, at least one geometric parameter of the IOL design iteration;
estimating, via the machine learning model, an updated predicted performance of the adjusted IOL design iteration; and
determining whether the performance threshold has been met based on the updated predicted performance of the adjusted IOL design iteration.

6. The system of claim 1, wherein the IOL design module is further configured to perform operations comprising:
generating multiple IOL design iterations;
predicting performances for each of the multiple IOL design iterations; and
selecting an IOL design based on a comparison of the predicted performances.

7. The system of claim 1, wherein the shape morphing engine employs topology modification algorithms to generate the IOL design iteration.

8. The system of claim 1, wherein the corresponding historical simulation data comprise results from physics-based computational simulations for individual IOL designs of the historical IOL design data, and wherein the machine learning model predicts the performance of the IOL design iteration based on learned relationships between geometric features extracted from the historical IOL design data and the results of the physics-based computational simulations.

9. The system of claim 1, wherein the IOL design module is further configured to perform operations comprising:
predicting an optical performance of the IOL design using the machine learning model.

10. A method for generating intraocular lens (IOL) designs, comprising:
training a deep learning model using historical IOL design data as an input and corresponding historical simulation data as an expected output;
after training the deep learning model, receiving input parameters including at least mechanical stability criteria for a new IOL design;
generating an IOL design iteration based on the input parameters using a shape morphing engine linked to the trained deep learning model;
predicting a performance of the IOL design iteration with respect to the mechanical stability criteria using the trained deep learning model;
iteratively refining the IOL design iteration using the shape morphing engine and based on the predicted performance; and
outputting the new IOL design based on the iterative refinement.

11. The method of claim 10, wherein the corresponding historical simulation data comprise results from physics-based computational simulations, and wherein the trained deep learning model predicts the performance of the IOL design iteration based on learned relationships between geometric features extracted from the historical IOL design data and the results of the physics-based computational simulations.

12. The method of claim 10, wherein generating the IOL design iteration comprises modifying an initial IOL design by adjusting, via the shape morphing engine automatically and without user intervention, at least one of an optic diameter, a haptic angle, an overall diameter, an edge thickness, or a surface curvature.

13. The method of claim 10, wherein the deep learning model comprises a neural network configured to extract geometric information from three-dimensional IOL designs.

14. The method of claim 10, wherein iteratively refining the IOL design iteration using the shape morphing engine and based on the predicted performance comprises:
determining whether a performance threshold has been met based on the predicted performance; and
in response to the performance threshold being met, selecting the IOL design iteration as the new IOL design; or
in response to the performance threshold not being met:
adjusting the IOL design iteration using the shape morphing engine; and
repeating the predicting and adjusting until the performance threshold is met.

15. The method of claim 10, wherein outputting the new IOL design comprises generating a three-dimensional model of the new IOL design.

16. The method of claim 10, wherein outputting the new IOL design comprises providing parametric descriptions of a geometry of the new IOL design.

17. A method for generating an intraocular lens (IOL) design with enhanced mechanical stability, comprising:
receiving historical IOL design data and corresponding historical simulation data;
training a machine learning model using the historical IOL design data as an input and the corresponding historical simulation data as an expected output;
receiving input parameters for a new IOL design, the input parameters including mechanical stability criteria;
generating multiple IOL design iterations based on the input parameters using a shape morphing engine;
predicting performances for each of the multiple IOL design iterations with respect to the mechanical stability criteria using the trained machine learning model;
selecting an IOL design iteration from the multiple IOL design iterations based on the predicted performances; and
outputting the selected IOL design iteration as the new IOL design.

18. The method of claim 17, wherein the mechanical stability criteria include a maximum allowable displacement under compression, and wherein generating the multiple IOL design iterations includes modifying at least one of an optic diameter, a haptic angle, an overall diameter, an edge thickness, or a surface curvature of a previous IOL design iteration.

19. The method of claim 17, wherein outputting the selected IOL design iteration as the new IOL design comprises:
generating a three-dimensional visualization of the new IOL design for display on a display device; and
storing the new IOL design to a non-transitory memory.

20. The method of claim 17, wherein selecting the IOL design iteration from the multiple IOL design iterations based on the predicted performances comprises:
ranking the multiple IOL design iterations with respect to at least the mechanical stability criteria; and
selecting a best-ranked IOL design iteration as the new IOL design.
